(19) United States
(12) Patent Application Publication
(10) Pub. No.: US 2015/0377211 A1
(43) Pub. Date: Dec. 31, 2015

(54) RENEWABLE ENERGY SYSTEM FOR GENERATING HYDROGEN AND HYDROGEN PRODUCTS

(71) Applicant: SAUDI BASIC INDUSTRIES CORPORATION, Riyadh (SA)

(72) Inventor: Ernesto OCCHIELLO, Riyadh (SA)

(73) Assignee: SAUDI BASIC INDUSTRIES CORPORATION, Riyadh (SA)

(21) Appl. No.: 14/767,537

(22) PCT Filed: Mar. 5, 2014
(86) PCT No.: PCT/IB2014/000906

§ 371 Date: Aug. 12, 2015

Related U.S. Application Data
(60) Provisional application No. 61/776,420, filed on Mar. 11, 2013.

(51) Int. Cl. F03D 3/00; C07C 29/00; F03D 9/00; C07C 1/02; C01C 1/02; C01B 3/02; C25B 1/04; C07C 5/02

(52) U.S. Cl. CPC F03D 3/005; C25B 1/04; C07C 29/00; C07C 5/02; C07C 1/02; C01C 1/02; C01B 3/025; F03D 9/002

(57) ABSTRACT

An apparatus for generating hydrogen includes a renewable energy source for generating electrical energy and a hydrogen generation module powered at least partially by electrical energy generated by the renewable energy source. The hydrogen generation module generates hydrogen through electrolysis of water. The hydrogen may be used by a chemical generation module to form hydrogen containing chemical products such as ammonia, methanol and/or an olefin. In a preferred embodiment, the renewable energy source comprises a high altitude wind system.

### CROSS REFERENCE TO RELATED APPLICATIONS

This application claims the benefit of U.S. Provisional Application No. 61/776,420 filed Mar. 11, 2013. The contents of the referenced application are incorporated into the present application by reference.

### BACKGROUND OF THE INVENTION

1. Field of the Invention

The present invention relates generally to renewable energy systems and more particularly to renewable energy systems for generating hydrogen that can be used to produce chemical products.

2. Description of Related Art

Systems for producing electrical energy from renewable energy sources are known. One disadvantage of renewable energy systems is that they may generate fluctuating amounts of electrical energy. For example, during times of low wind, a wind energy system may produce too little energy. Conversely, during times of high wind, a wind energy system may produce excess energy. Another disadvantage to wind energy systems is that prevailing winds are often located away from energy users and therefore require the construction and maintenance of energy transportation structures.

Accordingly, there is a need for a process and system that can utilize energy locally and is readily adaptable to the amount of energy produced.

### SUMMARY OF THE INVENTION

According to an exemplary embodiment of the present invention, a process for generating chemical products comprises generating electrical energy through a renewable energy source, generating hydrogen using, at least partially, electrical energy generated by the renewable energy source, and producing one or more chemical products utilizing the hydrogen generated using the electrical energy.

The renewable energy source may be generated by a high altitude wind system. The hydrogen may be generated using an electrolysis cell. The chemical product may comprise one or more of ammonia, methanol and/or an olefin.

According to another exemplary embodiment, an apparatus for generating chemical compositions comprises a renewable energy source for generating electrical energy, a hydrogen generation module powered at least partially by electrical energy generated by the renewable energy source, and a chemical generation module for processing generated hydrogen into chemical products.

The renewable energy source may be a high altitude wind system. The hydrogen generation module may be an electrolysis cell for generating hydrogen from water. The hydrogen module may use the chlor-alkali process to generate hydrogen. The chlor-alkali process may be one of a membrane, diaphragm or mercury process.

The chemical generation module may generate one or more of ammonia, methanol and/or an olefin. The chemical generation module may be an ammonia generation loop.

In accordance with another embodiment, a high altitude wind system for generating electrical energy and a hydrogen generation module powered at least partially by electrical energy generated by using airfoils to take advantage of high altitude winds.

The high altitude wind system may comprise a vertical-axis wind turbine, at least one kite disposed to be immersed in a wind current and connected to the vertical-axis wind turbine to rotate the vertical-axis wind turbine, and at least one generator cooperating with the vertical-axis wind turbine to generate electricity. The vertical-axis wind turbine may comprise an arm connected to the kite.

The high altitude wind system may comprise a carousel system that includes a circular rail and modules adapted to move on the rail. The system may contain a plurality of kites disposed to be immersed in a wind current and connected to modules which cause the modules to move on the rails and generate electricity based on the movement.

A processing module for using generated hydrogen to make at least one chemical product may be provided. The at least one chemical product may comprise ammonia, methanol and/or an olefin. The hydrogen generation module may comprise an electrolysis cell for generating hydrogen from water. The electrolysis cell may be a chlor-alkali electrolysis cell.

An auxiliary energy source for powering the hydrogen generation module may be provided and an energy storage device for storing excess energy may be provided.

A hydrogen storage device for storing excess hydrogen may be provided.

A floating platform for supporting the wind system and hydrogen generation module may be provided.

In another embodiment, a method for generating hydrogen comprises generating electrical energy using high altitude winds and generating hydrogen using the generated electrical energy. The hydrogen may be generated through electrolysis of water. The electrical energy may be generated using a kite wind energy system. The hydrogen may be reacted with other reactants to form a chemical product, and the chemical product may comprise ammonia, methanol and/or an olefin.

### BRIEF DESCRIPTION OF THE DRAWINGS

FIG. 1 is a schematic illustration of a renewable energy system in accordance with an illustrative embodiment of the invention;

FIG. 2 is a schematic illustration of an ammonia generation system in accordance with an illustrative embodiment of the invention; and

FIG. 3 is a schematic illustration of ammonia generation system in accordance with another illustrative embodiment of the invention.

### DESCRIPTION OF ILLUSTRATIVE EMBODIMENTS

In the following detailed description, reference is made to the accompanying drawings, in which are shown exemplary but non-limiting and non-exhaustive embodiments of the invention. These embodiments are described in sufficient detail to enable those having skill in the art to practice the invention, and it is understood that other embodiments may be used, and other changes may be made, without departing from the spirit or scope of the invention. The following detailed description is, therefore, not to be taken in a limiting sense, and the scope of the invention is defined only by the appended claims.

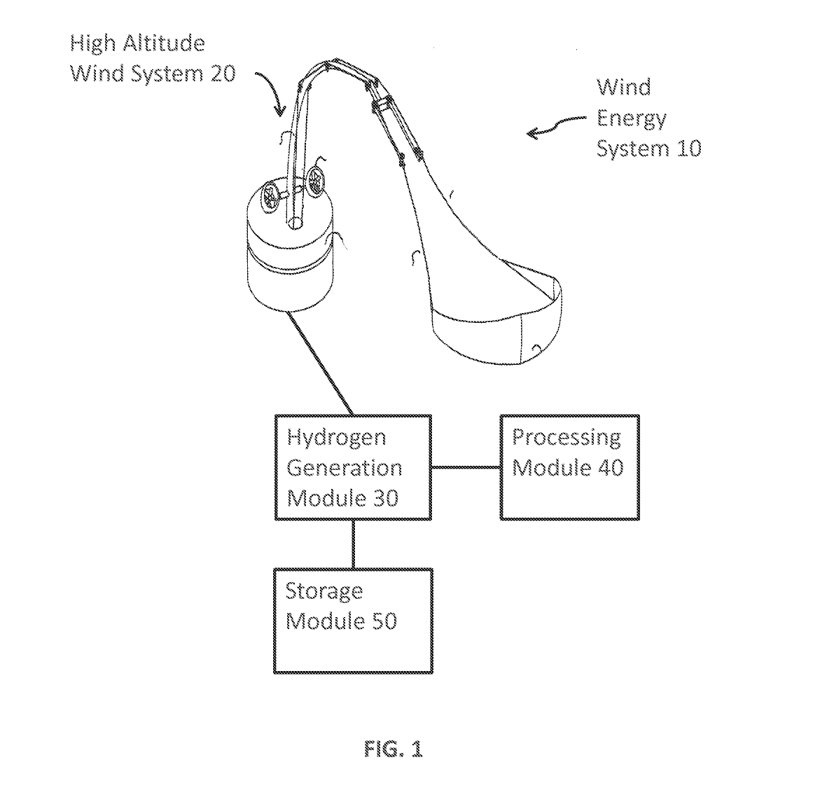

FIG. 1 shows a renewable energy system 10. The system includes a renewable energy source 20 for generating electrical energy and a hydrogen generation module 30 for generating hydrogen using the generated electrical energy. Preferably, the renewable energy source 20 and hydrogen generation module 30 are disposed at the same site (i.e., not connected by a conventional electrical grid). In one embodiment, at least one processing module 40 is included at the same site to utilize the hydrogen. The processing module 40 may use the hydrogen to produce chemical products such as ammonia, methanol, and/or olefins. A storage module 50 may be included to store excess generated power or excess generated hydrogen. Power or hydrogen can be stored until needed or until prices reach acceptable levels.

In the illustrated embodiment, the renewable energy system is a wind energy system. In other embodiments, the renewable energy source may also be a solar, rain, tidal, wave, or geothermal energy system.

In a preferred embodiment, the high altitude wind energy system is a kite wind energy system. One suitable kite wind energy system is disclosed in U.S. Pat. No. 8,080,889, which is incorporated by reference in its entirety. This patent describes a system for converting wind energy that has at least one power wing profile (i.e., a kite) which can be driven from the ground. The power wing profile is immersed in at least one aeolian current. A basic platform for controlling the wing profile and generating electric energy is placed at ground level and is connected through two ropes to the power wing profile. The basic platform is adapted to drive the wing profile and to generate electric energy. The two ropes are adapted to transmit forces from and to the wing profile and are used both for controlling a flight trajectory of the wing profile and for generating energy. The system described in this patent may be a mobile system.

Another suitable high altitude wind energy system is a vertical stem type kite wind energy system. One such vertical stem type system is described in European Patent No. 1672214B1, which is incorporated by reference in its entirety. This publication describes a wind energy system with, among other things, a movable stem used to raise the kite to an initial take-off height, a guiding system that expands a kite to an open position, and winches that are automatically controlled by a kite piloting system. Another such vertical stem type system is described in U.S. Patent Publication 2011/0074161, which is incorporated by reference in its entirety. This publication describes a wind energy system with, among other things, an orientable arm connected by control cables to a wing (i.e., a kite). A jet-type ventilation plant is provided to assist with initial take-off of the wing. A variable-geometry system and a driving system are provided for controlling and supporting the wing. Such a system may be located on-shore or off-shore (i.e., on a floating platform).

Yet another suitable high altitude wind energy system kite system is vertical axis system described in U.S. Pat. No. 8,134,249 B2, which is incorporated by reference in its entirety. This patent discloses a wind energy system that utilizes a plurality of kites connected to a rotatable arm. The rotatable arm turns about a vertical axis and is geared with a conventional generator. The vertical axis system provides certain advantages, especially with respect to takeoff of the kites. Such a carousel system may be located on-shore or off-shore (i.e., on a floating platform).

Further examples of suitable high altitude wind energy systems are described in the following references, each of which is incorporated by reference in its entirety. U.S. Pat. No. 8,319,368 discloses carousel system equipped with a circular rail where movement of the models resulting from traction pull from the kites, causes the modules to move on the rails and generate electricity based on the movement. U.S. Pat. No. 4,124,182 discloses a device equipped with “parakite” (or “modified parachute”) to capture aeolian energy and convert it into a rotary motion of a shaft which actuates a generator. This device is characterized by a pair of “trains of parakite” in which the wing profiles are arranged in series. Chinese patent CN 1,052,723 discloses an aeolian current generator equipped with a pair of kites through which the fraction exerted by the aeolian currents is converted, through high-resistance ropes, into rotation of a drum placed at ground level. British patent GB 2,317,422 discloses a device equipped with multiple wing profiles which, due to the effect of wind action, rotate a vertical shaft connected to a generator for producing current. U.S. Pat. No. 6,072,245 discloses a device for exploiting aeolian energy composed of multiple kites connected to ropes which form a ring. The kites are driven in order to alternate an ascending path and a descending path determining a rotary ring motion always along the same direction. U.S. Pat. No. 6,254,034 discloses a device equipped with a wing profile (“tethered aircraft”) pushed by the aeolian currents at a controlled speed, in order to exploit the wind aeolian energy. Dutch patent NL 1017171C discloses a device similar to the previously described one in which however the manual driving mode is not provided, and in which the recovery of the wing profile occurs by inclining the kite as a flag, in order to minimize the wind thrust when rewinding the ropes. U.S. Pat. No. 6,523,781 discloses a device composed of a wing profile (“airfoil kite”) through which the aeolian energy is captured, having an entry edge, an exit edge and two side edges. United States application US2005046197 discloses a device equipped with a wing profile (“kite”) for exploiting the aeolian energy which generates electricity by actuating, by means of ropes, a winch connected to a generator.

In addition to the renewable energy source, an auxiliary energy source can be provided to provide a steady state supply of electricity as needed by the plant/site to account for variations in wind energy. The auxiliary energy source can include transmission lines connected to an electrical grid, fossil fuel plants, nuclear power plants, another renewable energy source or any other conventional energy source.

The hydrogen generator 30 generates hydrogen through electrolysis of water. Preferably, the hydrogen generator is able to quickly respond to dynamic changes in the amount of available electricity. One suitable hydrogen generation module is the NEL P•60 pressurized electrolyzer, which is available from NEL Hydrogen AS, Notodden, Norway. Another suitable hydrogen generation module is a polymer electrolyte membrane electrolyzer such as those developed by Siemens AG, Munich, Germany. A membrane electrolyzer is advantageous in that it reacts to changes in available electric energy in milliseconds.

The hydrogen generator 30 may also be a chlor-alkali plant. The chlor-alkali process is an energy intensive process for the electrolysis of sodium chloride solutions. In addition to hydrogen, the chlor-alkali process creates other useful products, including chlorine and sodium hydroxide. Preferably, the chlor-alkali plant utilizes a membrane cell. Other types of chlor-alkali plants such as a diaphragm cell and mercury cell plants may be used as well. The chlorine produced by the chlor-alkali process may be liquefied and stored on site for future use or for transportation.

The hydrogen may be stored for further processing or for transportation. In one embodiment, at least one processing module 40 is included at the same site to utilize the hydrogen. The processing module 40 may use the hydrogen to produce chemical products such as ammonia, methanol, and/or olefins. The hydrogen may be transported by ground transportation or by pipeline.

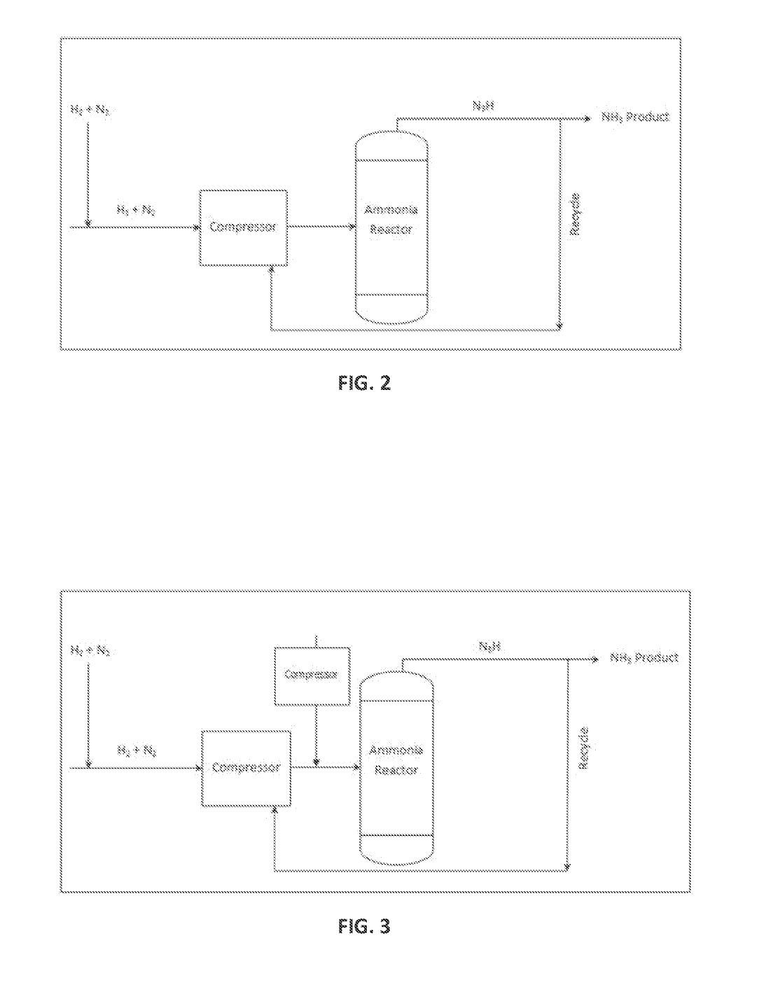

FIGS. 2 and 3 are schematic illustrations of suitable ammonia generation system which utilize hydrogen. In these figures, the ammonia generation plant is a continuous running plant. In the embodiment of FIG. 2, additional hydrogen generated by the renewable energy source and nitrogen are added upstream of the synthesis-loop compressor. In the embodiment of FIG. 3, additional hydrogen generated by the renewable energy source and nitrogen are added downstream of the synthesis-loop compressor.

Both of these systems will allow up to about 30% additional flow of hydrogen. Ammonia reactors typically operate at around an average maximum of 430° C. temperature and 210 bar pressure. The reactors are normally not a bottleneck in turn down ratios of the plant. The main bottleneck in the turn down ratio is the synthesis compressor which boosts synthesis loop pressure from around 30 to 210 bar. For continuous operation of an ammonia loop, the minimum load should be around 70% for efficient operation. This figure can be reduced but in this case kick-backs of compressor have to be opened to prevent surges.

In addition to the above methods, which utilize extra hydrogen in existing ammonia production, an independent synthesis loop can be set up to process hydrogen. An independent loop may be costly, however, and if additional hydrogen is not continuously available then a restart of reactor would require additional time 4-12 hours depending on the duration of shut down.

A storage module 50 may be included to store excess generated power or excess generated hydrogen. Power or hydrogen can be stored until needed or until prices reach acceptable levels. The hydrogen storage may take the form of gaseous hydrogen storage. Gaseous hydrogen storage is extensively employed around the world for both large and small scale storage. The two main methods currently used for large-scale hydrogen gas production are cavities created by dissociation in salt formations and deep aquifer layers. One example of a gaseous hydrogen cavern is located at Teesside, UK and operated by Sabic Petrochemicals. It has a 3×70,000 cubic meter storage capacity.

The disclosed system advantageously converts electricity into a marketable product other than electricity. This allows for a more efficient use of resources (to some degree a self-contained system for producing chemical products). The system could be used to produce hydrogen but any excess could be further utilized in the plant/site for other purposes.

1.-13. (canceled)

14. An apparatus comprising:
a high altitude wind system for generating electrical energy comprising:
a vertical-axis wind turbine;
at least one kite disposed to be immersed in a wind current, the at least one kite connected to the vertical-axis wind turbine to rotate the vertical-axis wind turbine; and
at least one generator cooperating with the vertical-axis wind turbine to generate electricity; and
a hydrogen generation module powered at least partially by electrical energy generated by the high altitude wind system.

15. (canceled)

16. The apparatus of claim 14, wherein the vertical-axis wind turbine comprises an arm connected to the kite.

17. The apparatus of claim 14, wherein the high altitude wind system comprises:
a vertical-axis wind turbine comprising a carousel; and
a plurality of kites disposed to be immersed in a wind current, the plurality of kites being connected to the carousel to rotate the vertical-axis wind turbine.

18. The apparatus of claim 14, further comprising a processing module for using generated hydrogen to make at least one chemical product.

19. The apparatus of claim 18, wherein the at least one chemical product comprises ammonia, methanol and/or an olefin.

20. The apparatus of claim 14, wherein the hydrogen generation module comprises an electrolysis cell for generating hydrogen from water.

21. The apparatus of claim 20, wherein the electrolysis cell is a chlor-alkali electrolysis cell.

22. The apparatus of claim 14, further comprising an auxiliary energy source for powering the hydrogen generation module.

23. The apparatus of claim 14, further comprising an energy storage device for storing excess energy.

24. The apparatus of claim 23, further comprising a hydrogen storage device for storing excess hydrogen.

25. The apparatus of claim 14, further comprising a floating platform for supporting the wind system and hydrogen generation module.

26. A method for generating hydrogen, comprising:
generating electrical energy using a high altitude wind system comprising:
a vertical-axis wind turbine;
at least one kite disposed to be immersed in a wind current, the at least one kite connected to the vertical-axis wind turbine to rotate the vertical-axis wind turbine; and
at least one generator cooperating with the vertical-axis wind turbine to generate electricity; and
generating hydrogen using the generated electrical energy.

27. The method of claim 26, wherein the hydrogen is generated through electrolysis of water.

28. (canceled)

29. The method of claim 26, further comprising combining the hydrogen with other reactants to form a chemical product.

30. The method of claim 29, wherein the chemical product comprises ammonia, methanol and/or an olefin.

31. The method of claim 26, wherein hydrogen is generated using a chlor-alkali process.

32. The method of claim 31, wherein the chlor-alkali process is one of a membrane, diaphragm or a mercury process.
